(19) United States
(12) Patent Application Publication
(10) Pub. No.: US 2026/0345265 A1
(43) Pub. Date: Nov. 5, 2026

(54) Perioperative Cobotic System with Surgeon-Specific Workflow

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/432,215

(22) Filed: Dec. 24, 2025

Related U.S. Application Data
(63) Continuation-in-part of application No. 19/195,731, filed on Apr. 30, 2025.

(51) Int. Cl. A61B 34/32; A61B 34/00; A61B 90/00; G06F 3/01; G06F 3/16; G06Q 10/0639; G16H 10/60; G16H 40/20

(52) U.S. Cl. CPC A61B 34/32; A61B 34/25; A61B 90/03; G06F 3/017; G06F 3/167; G06Q 10/06398; G16H 10/60; G16H 40/20; A61B 2034/258

(57) ABSTRACT

A perioperative cobotic system is provided for executing surgeon-specific workflows in an operating room environment. The system includes a configurable cobotic platform with one or more articulated robotic arms, tool handling interfaces, and surgical-environment sensors. A workflow engine retrieves and applies personalized surgeon profiles defining instrument preferences, procedural sequencing, ergonomic positioning, and timing cues, based on surgeon identification. An instrument handling module positions tools and configures the cobot in accordance with the selected profile. An adaptive learning engine refines workflow profiles using intraoperative data and surgeon feedback over time. Integration with electronic medical records and scheduling systems enables procedure- and patient-specific profile adjustments. A context-aware execution module responds to voice, gesture, or footswitch inputs and detected surgical phases. Safety, override, and logging features support compliant operation and performance analytics.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by refer

### TECHNICAL FIELD

The present invention relates generally to surgical robotics and perioperative workflow optimization, and more particularly to cobotic systems configured to execute personalized, surgeon-specific workflows.

### BACKGROUND

Modern surgical procedures demand high levels of precision, efficiency, and coordination between surgical personnel and supporting systems. Despite advancements in robotic surgery and perioperative automation, most current systems lack the ability to adapt to the nuanced preferences and procedural styles of individual surgeons. Standardized workflows often fail to accommodate variability in instrument usage patterns, ergonomic preferences, and decision-making sequences, which can lead to workflow friction, increased cognitive load, and procedural inefficiencies.

Existing robotic systems generally operate under generic configurations or pre-programmed routines that are detached from a surgeon’s personal habits. They typically do not account for how different surgeons arrange their instruments, sequence their procedural steps, or interact with tools during a case. As a result, these systems may require manual intervention, introduce delays, or create inconsistencies in surgical performance.

Furthermore, current systems rarely leverage data collected across multiple procedures to refine future workflow performance. There is little support for learning from past cases, adapting to surgeon-specific timing and handoff cues, or updating instrument sequencing based on historical behavior. Even when electronic medical records (EMRs) and OR scheduling systems are integrated into the surgical workflow, these data streams are often underutilized, especially in real-time intraoperative contexts.

Another limitation lies in the lack of flexible, intuitive interfaces that allow surgeons to easily customize and approve their workflow preferences prior to surgery. In many instances, workflow adjustments must be manually relayed to clinical support staff, increasing the risk of miscommunication and variability. Additionally, most perioperative systems lack robust safety and compliance protocols for managing emergency overrides, role-based access control, or logging of intraoperative workflow deviations for postoperative review and quality assurance.

Accordingly, there exists a need for an intelligent, surgeon-adaptive cobotic platform that can retrieve, execute, and refine personalized workflow profiles, integrating EMR and scheduling data, responding dynamically to intraoperative conditions, and improving over time through adaptive learning. Such a system would improve workflow efficiency, support ergonomic surgeon preferences, enhance intraoperative coordination, and ultimately contribute to safer, higher-quality surgical outcomes.

### SUMMARY

The present disclosure relates to a perioperative cobotic system configured to execute and refine surgeon-specific workflows. The system includes a configurable cobotic platform comprising one or more articulated robotic arms, tool handling interfaces, and surgical environment sensors. The platform is designed to operate safely within the operating room, autonomously navigating and interacting with the environment while avoiding sterile zones and responding to real-time procedural changes.

A surgeon-specific workflow engine retrieves and executes personalized workflow profiles based on unique surgeon identifiers, such as RFID badges or biometric data. Each profile defines instrument preferences, procedural sequences, ergonomic handoff positioning, and timing cues. The system incorporates an instrument handling and pre-positioning module that arranges tools and configures cobot positioning according to the selected profile.

An adaptive learning engine updates workflow profiles over time by collecting intraoperative data and integrating explicit surgeon feedback. The system interfaces with hospital EMR and OR scheduling systems to pre-load or adjust profiles in alignment with the scheduled procedure and patient context.

A context-aware task execution module detects surgeon commands, via voice, gesture, or footswitch, and dynamically adapts cobotic behavior in response to surgical phase detection and environmental cues. A user-facing interface enables preoperative review and customization of surgeon preferences, supporting both global defaults and procedure-specific overrides.

The system includes safety, compliance, and override mechanisms to ensure sterile protocol adherence, enforce role-based access controls, and log workflow actions for postoperative analysis and continuous quality improvement. An integrated analytics and reporting module captures surgeon-specific performance data and generates reports for surgical leadership and administrative oversight.

### BRIEF DESCRIPTION OF DRAWINGS

The foregoing and other objects, features, and advantages of the invention will become more apparent from the following detailed description, taken in conjunction with the accompanying drawings, in which:

FIG. 1 is a perspective view of the perioperative cobotic system, including the mobile base, articulated arm, instrument tray, and interaction zone.

FIG. 2 is a schematic block diagram of the system architecture, illustrating the workflow engine, instrument handling module, adaptive learning engine, and context-aware control components.

FIG. 3 is a diagram showing profile-based cobot configuration and instrument arrangement according to a selected surgeon’s workflow preferences.

FIG. 4 is a flowchart depicting the process of retrieving a surgeon-specific workflow profile and configuring the cobotic system for intraoperative execution.

FIG. 5 is a flow diagram of the adaptive learning engine, illustrating profile refinement through data capture, procedural analysis, and feedback integration.

FIG. 6 is a diagram illustrating dynamic integration of EMR and OR scheduling inputs into the workflow selection and configuration process.

FIG. 7 is a diagram showing context-aware cobotic actions triggered by surgeon inputs and phase-based task sequencing.

FIG. 8 is a user interface view showing the surgeon preference editor with procedure-specific workflow visualization and customization tools.

FIG. 9 is a block diagram of safety and override subsystems, including emergency stop controls, role-based access enforcement, and workflow event logging.

FIG. 10 is a report generation diagram illustrating how surgeon-specific performance data is aggregated, analyzed, and presented to hospital stakeholders.

### DETAILED DESCRIPTION

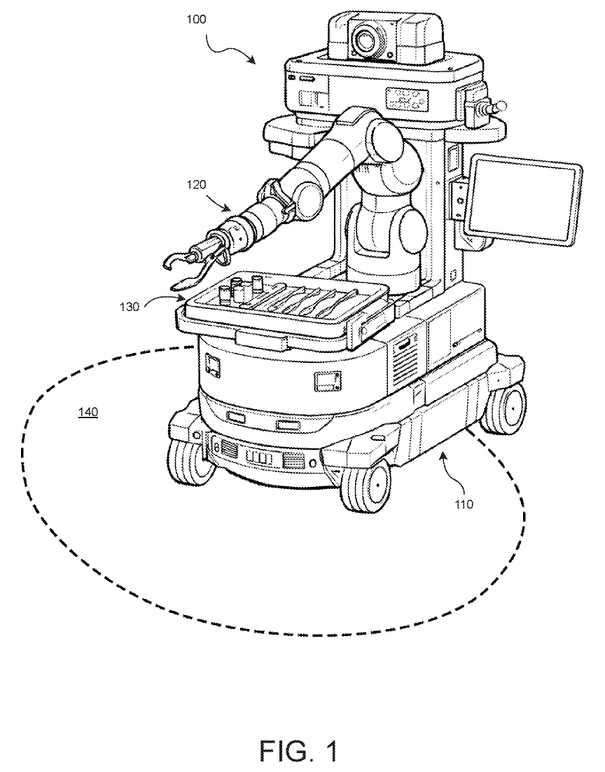

Embodiments of the present disclosure are described below with reference to FIGS. 1-10. These examples are presented to illustrate one or more implementations of the perioperative cobotic system with surgeon-specific workflow and to enable those skilled in the art to practice the system, but they are not intended to be limiting. For clarity and consistency, like reference numerals refer to like components across the figures where applicable.

As shown in FIG. 1, an exemplary perioperative cobotic system 100 includes a mobile base 110, an articulated robotic arm 120, an instrument tray 130, and a defined interaction zone 140 for surgeon engagement. The mobile base 110 supports the system’s structure and houses locomotion components (e.g., wheels or omni-directional casters) and a power source, enabling the cobot to move or be positioned as needed around the operating room. In some embodiments, the base 110 is semi-fixed or floor-mounted but still allows repositioning between surgical stations. The arm 120 is mounted on the base and is highly articulated, providing multiple degrees of freedom to reach different locations in the surgical field and to present or retrieve instruments. The entire platform 100 is designed for the sterile environment: its surfaces are smooth and easy to sterilize, and it is equipped with sensors to detect obstacles and human presence so that it can navigate safely without colliding with staff or equipment.

The instrument tray 130 is attached to the system (for example, mounted on the base 110 or on the arm 120) and is used to hold surgical instruments in an organized manner. The tray 130 is configured to be accessible both to the robotic arm 120 and to the surgeon. The interaction zone 140 is a designated area where the cobot and the surgeon exchange instruments and interact. For instance, the interaction zone 140 may be a region directly above the instrument tray or adjacent to the arm’s workspace where the cobot can safely hand over an instrument to the surgeon (and vice versa) without breaching sterile field protocols. This zone 140 is defined through software and sensors so that the cobot’s motions remain confined to a safe handoff area, it ensures that when the cobot presents an instrument, it does so within the surgeon’s comfortable reach and without entering any restricted sterile area around the

patient. Overall, FIG. 1 provides a physical overview of the system’s main components and their arrangement, demonstrating a platform built to adapt to the operating room setting while maintaining sterility and allowing close cooperation with human surgeons.

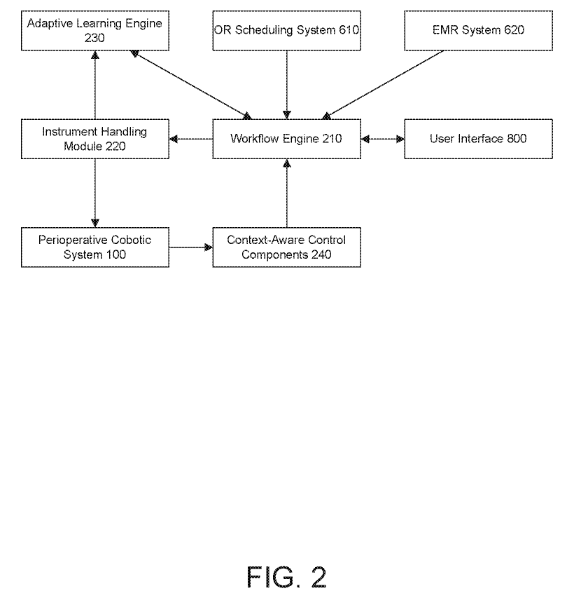

FIG. 2 illustrates a high-level architecture of the system, which is organized into several modules that collectively manage the cobot’s operations.

Workflow Engine 210: This is the central control software responsible for orchestrating the overall workflow according to the selected surgeon-specific profile. It coordinates the timing of actions and the communication between other modules. For example, the workflow engine 210 determines when the next instrument should be prepared and signals the instrument handling module 220 accordingly.

Instrument Handling Module 220: The instrument handling module controls the cobot’s physical interactions with surgical tools, specifically managing the robotic arm 120 and instrument tray 130. It executes the pick-up, positioning, and handoff of instruments as instructed by the workflow engine, using real-time sensor input to ensure precision and safety during each movement.

Adaptive Learning Engine 230: This module monitors system performance and outcomes over time, analyzing data from each procedure to refine the surgeon’s profile. The adaptive learning engine 230 uses machine learning or rule-based algorithms to identify patterns (such as frequently used instruments or timing adjustments) and can suggest or implement updates to the workflow engine’s parameters for future surgeries.

Context-Aware Control Components 240: These components include sensors and processing algorithms that give the cobot awareness of the surgical context. The context-aware control monitors inputs like visual sensors, instrument usage, and surgeon cues, and adjusts the cobot’s behavior in real time. For instance, if the surgeon changes pace or an unexpected event occurs, this module might signal the workflow engine 210 and instrument handling module 220 to adapt (such as slowing down an instrument delivery or pausing a task).

These modules communicate with each other and with external data sources (such as surgeon profile databases and hospital systems) to ensure the cobot executes tasks in accordance with the active surgeon-specific workflow profile.

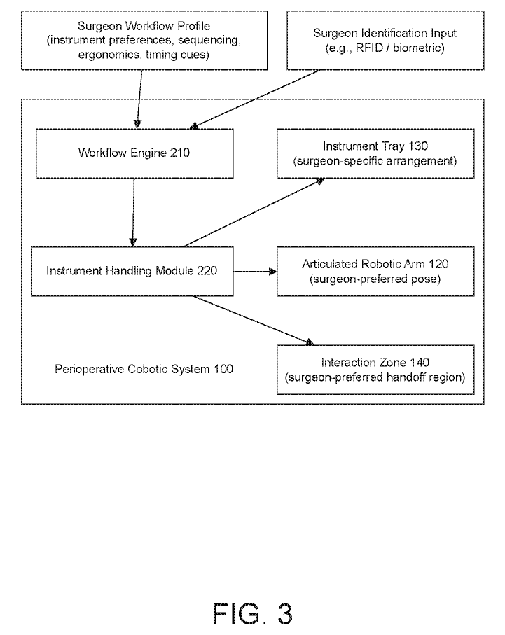

FIG. 3 shows how the system 100 configures itself based on a selected surgeon’s workflow profile. When a particular surgeon’s profile is active, the cobot adjusts various settings to match that surgeon’s established preferences. For example, the instrument tray 130 can be

arranged with instruments placed in specific positions or order that the surgeon prefers. If Surgeon A typically uses a certain instrument as the first tool and keeps it on the far left of the tray, the system will ensure that instrument is placed accordingly when Surgeon A’s profile is loaded. Similarly, the articulated arm 120 may move to a default resting position or angle that the surgeon finds most convenient for initial instrument handoff. Profile-specific configuration can include adjusting the height or tilt of the tray 130 and the boundaries of the interaction zone 140 to accommodate the surgeon’s dominant hand or comfort zone. For instance, a left-handed surgeon’s profile might direct the system to position the tray slightly to the left of center and present instruments to the left side of the interaction zone 140. By contrast, a different surgeon’s profile could result in a different layout and orientation (as indicated schematically in FIG. 3 by alternate instrument placements), illustrating that the cobot dynamically adapts its physical configuration (both the tool arrangement and how it positions its arm) to align with individual workflow styles. This profile-based configuration reduces the cognitive load on the surgeon, since instruments and robot positioning will feel familiar and expected for each surgeon using the system.

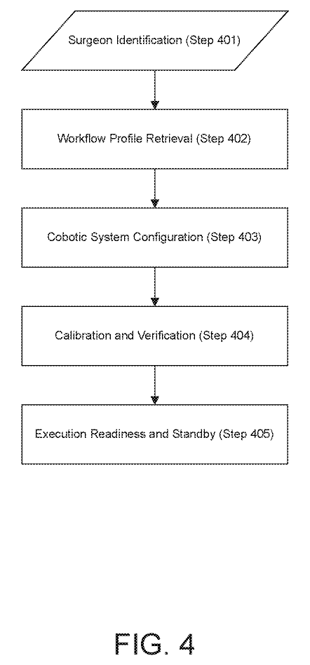

FIG. 4 is a flowchart depicting an example process for loading a surgeon-specific workflow profile and preparing the perioperative cobotic system for use.

Surgeon Identification (Step 401). The system identifies the operating surgeon by obtaining a surgeon identifier. The identifier may be acquired by reading an RFID badge, detecting a biometric login, receiving credentials via a user interface, or retrieving surgeon identity information from operating room scheduling data. This step establishes which surgeon-specific workflow profile is to be accessed.

Workflow Profile Retrieval (Step 402). The workflow engine 210 retrieves the workflow profile corresponding to the identified surgeon from a profile data store 205. The workflow profile includes surgeon-specific preferences such as instrument selection, procedural sequencing, instrument handoff timing, ergonomic positioning parameters, and interaction zone preferences.

Cobotic System Configuration (Step 403). Based on the retrieved workflow profile, the cobotic system configures one or more system components. The instrument handling module 220 arranges instruments on the instrument tray 130 according to the specified order and placement. The articulated robotic arm 120 is positioned to a surgeon-preferred starting pose,

and the interaction zone 140 is defined or adjusted to align with the surgeon’s expected handoff location and reach envelope.

Calibration and Verification (Step 404). The system performs calibration, safety checks, and verification routines. These routines may include confirming the presence and location of required instruments, validating reachable motion paths of the articulated arm 120, verifying sterile-field boundaries, and executing system self-tests. Upon successful verification, the system generates a readiness indication and may prompt the surgeon or operating room staff to confirm the configuration.

Execution Readiness and Standby (Step 405). After successful configuration and verification, the cobotic system transitions to an active standby state in which it is prepared to execute the surgeon-specific workflow during the surgical procedure, including presenting and exchanging instruments, adjusting positions, and responding to surgeon inputs in accordance with the loaded workflow profile.

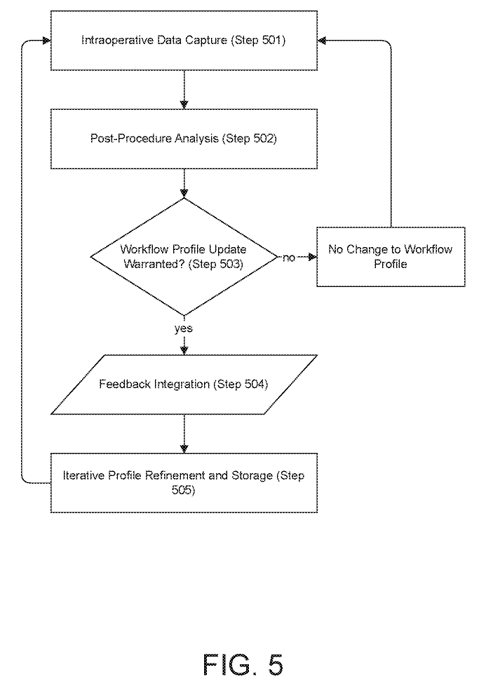

FIG. 5 is a flow diagram illustrating how the adaptive learning engine 230 refines a surgeon-specific workflow profile over time through continuous learning.

Intraoperative Data Capture (Step 501). During a surgical procedure, the system captures operational data associated with workflow execution. Captured data may include timestamps of instrument requests and handoffs, deviations from expected procedural sequences, manual interventions by the surgeon or staff, sensor data, and context-aware events detected during the procedure.

Post-Procedure Analysis (Step 502). Following completion of the procedure, the adaptive learning engine 230 analyzes the captured data by comparing actual intraoperative events to the expected workflow defined by the surgeon’s profile. The analysis identifies recurring patterns, timing variations, inefficiencies, or deviations.

Workflow Profile Adjustment Determination (Step 503). Based on the analysis, the adaptive learning engine 230 determines whether updates to the workflow profile are warranted. Potential adjustments may include modifying instrument sequencing, adjusting predicted timing for handoffs, or updating readiness cues, with certain adjustments applied automatically within predefined safety constraints.

Feedback Integration (Step 504). Explicit feedback provided by the surgeon or authorized operating room personnel is incorporated into the refinement process. The adaptive

learning engine 230 weights such feedback to ensure surgeon-directed preferences guide or override automated inferences where appropriate.

Iterative Profile Refinement and Storage (Step 505). The updated workflow profile is stored and associated with the surgeon for subsequent procedures. Steps 501 through 504 are repeated over multiple procedures, enabling iterative refinement of the workflow profile so that the cobotic system becomes progressively aligned with the surgeon’s individual workflow.

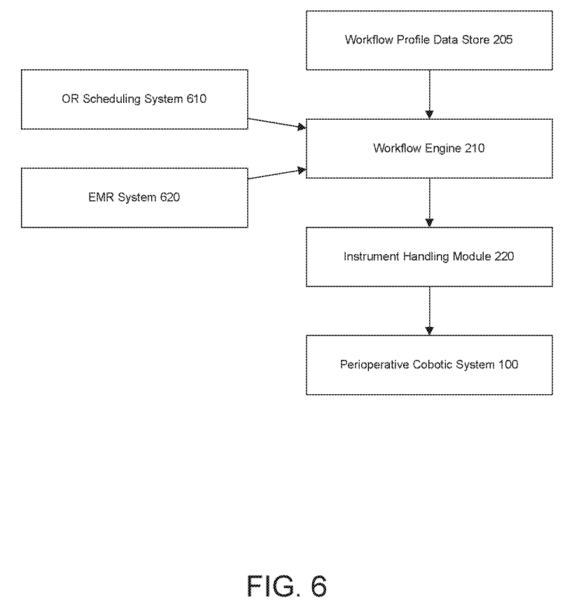

FIG. 6 illustrates the integration of the cobotic system with hospital information systems to streamline setup and configuration. The workflow engine 210 is communicatively connected to an OR scheduling system 610 and an electronic medical records (EMR) system 620 (or similar hospital database). Prior to a scheduled surgery, the OR scheduling system 610 provides the cobot with relevant information such as the identity of the surgeon performing the procedure, the type of surgery, and the scheduled time and location. Using this information, the workflow engine 210 can automatically select and pre-load the appropriate surgeon-specific profile (as described in FIG. 4) so that the system is configured well before the operation begins. Additionally, the EMR system 620 can supply patient-specific data or surgical notes that might affect the workflow. For example, if the patient has an allergy that necessitates a special surgical tool or if the procedure plan indicates an uncommon step, the system can ensure those considerations are incorporated (such as including the special tool in the tray or adjusting the workflow sequence). By integrating with these external systems, the cobot can adapt its configuration on-the-fly: if there is a last-minute change (for instance, a different surgeon is assigned or the procedure plan is modified), the workflow engine 210 receives the update from scheduling or EMR and loads the corresponding profile or adjustments immediately. FIG. 6 thus demonstrates how automation through EMR and scheduling integration helps the cobot arrive at the correct setup with minimal manual input, improving efficiency and reducing the risk of human error in configuring the system for each surgery.

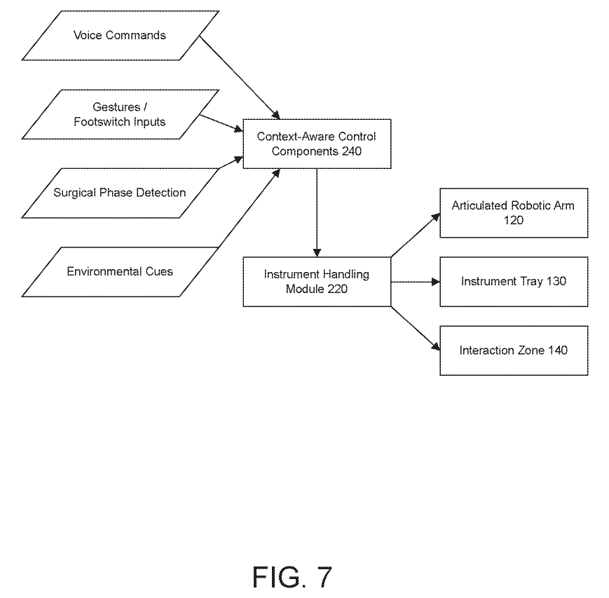

FIG. 7 depicts how the cobot responds intelligently to real-time inputs and situational cues during surgery. The context-aware control components 240 continuously monitor both direct surgeon commands and the overall surgical context to trigger appropriate actions. Several examples are discussed below.

Voice Commands: The system is configured to recognize surgeon voice instructions (e.g., the surgeon saying the name of an instrument or giving a command like “next”). In response to a recognized command, the cobot’s instrument handling module 220 promptly brings the

requested instrument to the surgeon via the interaction zone 140. For example, if the surgeon says “Scalpel,” the system identifies this command and the instrument handling module 220 locates the scalpel on tray 130, then the arm 120 presents it in the correct orientation for the surgeon to grasp safely.

Gestures or Footswitch Inputs: The cobot can interpret certain hand gestures or the press of a footswitch as cues for action. As an illustration, if the surgeon extends an open hand toward the tray 130, the system (via cameras or proximity sensors) detects this gesture as a request for the next instrument. The context-aware control 240 then signals the instrument handling module 220 to place the next instrument into the surgeon’s hand. Similarly, a foot pedal press could be mapped to a specific function, such as toggling a suction device or instructing the cobot to retract an instrument. The key is that the cobot observes the surgeon’s non-verbal cues and assists accordingly, which reduces the need for the surgeon to break focus or verbally call out every minor request.

Surgical Phase Detection: The system also employs algorithms to deduce the current phase of the surgery by analyzing data like instrument usage patterns, timing, or live video feed. When the system detects that the procedure has entered a new phase (for example, transitioning from tumor resection to suturing), the context-aware module 240 can automatically adjust the cobot’s behavior. In practice, this means the cobot anticipates the needs of the new phase, it might prepare suturing instruments, position the robotic arms and remove resection tools from the tray as the closure phase begins. By proactively adapting in sync with surgical phases, the cobot ensures that the right tools are ready at the right time without needing explicit instructions for routine transitions.

Environmental Cues: The cobot is also aware of changes and events in the operating room environment. If an unexpected situation arises, for instance, if a surgeon or nurse manually takes an instrument from the tray without the cobot’s involvement, or if a patient’s condition causes the surgery to pause, the system recognizes these events through its sensors. In such cases, the cobot might temporarily halt its current action and enter a standby state to avoid interference. Once normal activity resumes (e.g., the surgeon is ready to continue), the cobot picks up where it left off, perhaps re-confirming the current step with a voice prompt or simply resuming instrument handoffs as appropriate. This context awareness ensures the robotic assistant remains helpful and non-disruptive, adjusting its timing and behavior so that it complements the surgical team’s workflow under all circumstances.

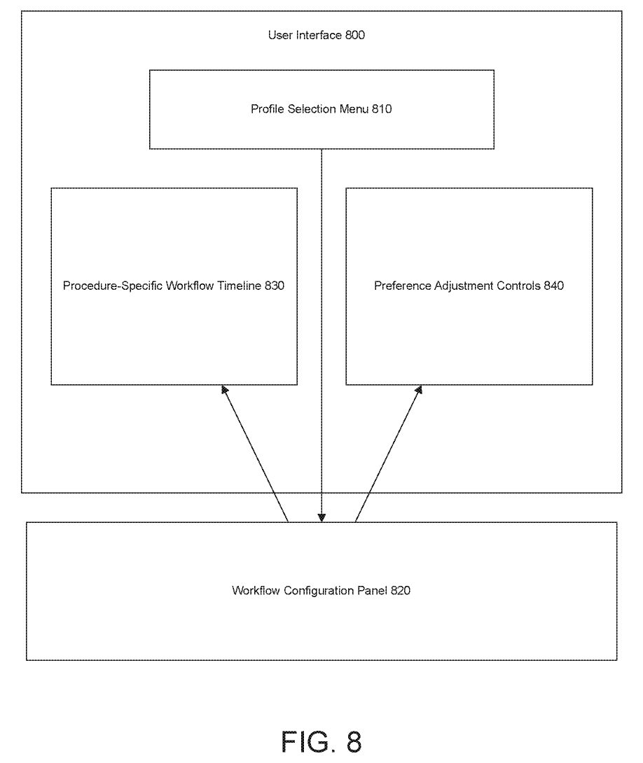

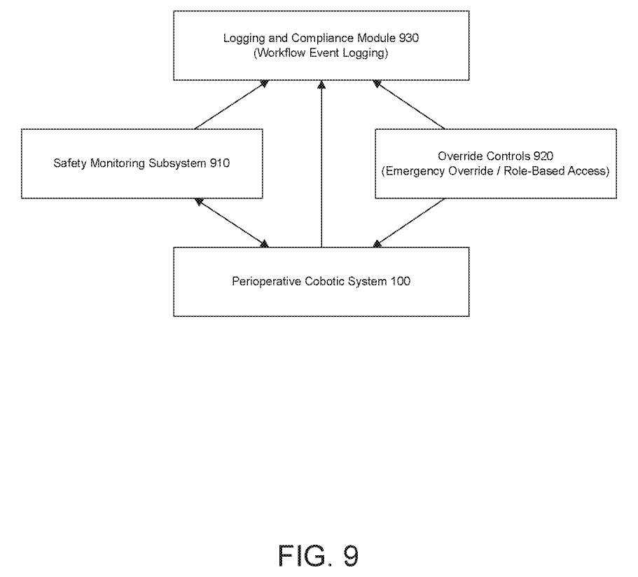

FIG. 8 provides an example of a user interface (UI) 800 through which surgeons and authorized staff can view and customize workflow profiles. The interface 800 allows selection of a surgeon’s profile from a list or menu 810, and it may allow filtering or choosing specific procedure templates under that surgeon’s name. Upon selecting a profile, the UI displays detailed configuration information in a panel 820. This panel 820 visualizes the surgeon’s preferred instrument sequence and timing in an intuitive format, for example, it could show a timeline 830 or flow diagram of the surgical procedure, annotated with icons or names of instruments at the points they are used. The UI 800 enables the user to modify the profile by interacting with these elements: the user might drag and drop instruments on the timeline 830 to change the order, or click on an instrument to adjust a setting (such as “present this instrument earlier/later”). Other preference settings 840 can also be adjusted, such as the position of the instrument tray 130 relative to the patient (for ergonomic comfort) or the default speed at which the cobot hands over instruments. The interface includes safeguards to prevent unintended changes, for instance, making substantial modifications might require entering an administrator password or the surgeon’s confirmation (ensuring role-based access control as mentioned in FIG. 9). The UI 800 may also highlight differences between a global profile (the surgeon’s default for all procedures) and a procedure-specific profile; the user can toggle views to see which settings are universally applied and which are overridden for a particular procedure type. After customization, the updated profile is saved to the system’s database and will be utilized by the workflow engine 210 in subsequent surgeries. FIG. 8 thus illustrates the system’s means for making the workflow profiles transparent and editable, giving human users intuitive oversight over the automated workflow definitions.

FIG. 9 highlights the subsystems responsible for ensuring safe operation and oversight of the cobotic system. These include:

Safety Monitoring Subsystem 910: A dedicated safety controller continuously monitors the cobot’s sensors and movement to prevent any unsafe conditions. This subsystem enforces rules such as maintaining appropriate distances from the patient and staff, respecting the sterile field boundaries, and limiting the force and speed of the arm 120 during instrument handoff. If the cobot’s sensors (e.g., proximity detectors or force sensors) detect a potential collision or a boundary breach, for example, if the arm 120 is moving too close to the surgeon or a sterile area, the safety subsystem 910 can intervene by stopping or slowing down the motion. It can also emit warnings or notifications to alert the operator when needed. These measures ensure

that the introduction of robotic assistance does not compromise the strict safety and sterility requirements of the operating room.

Override Controls 920: The system provides manual override mechanisms that allow human team members to instantly assume control or halt the cobot’s actions. For instance, emergency stop buttons can be mounted on the cobot or provided on a remote interface; pressing an emergency stop immediately freezes all robotic motion. Additionally, the system’s control software can feature an override mode in which the cobot yields to manual operation, in this mode, a human can physically reposition the arm 120 or manually handle instruments without resistance. The design includes a level of user privilege management (role-based access) for such overrides: only authorized personnel (such as the lead surgeon or a designated technician) can activate certain override functions or make permanent changes to the system’s configuration. This prevents accidental or malicious interruption of the workflow by unauthorized users while still ensuring that in any critical situation, the surgical team can directly control the system.

Logging and Compliance Module 930: All activities of the cobot are tracked by a logging subsystem for quality assurance and compliance purposes. This module records a timeline of events such as which instrument was handed off at what time, any voice commands recognized and executed, any adjustments made by the adaptive learning engine 230, and all safety or override events (like when an emergency stop was pressed or when the safety subsystem halted an action). These logs create an audit trail that can be reviewed after the surgery to assess performance or investigate any irregularities. They also serve to document that the system adhered to safety protocols (useful for compliance with hospital policies or regulatory standards). In addition, the logging data feeds back into the system’s learning and maintenance processes: the recorded information can be analyzed to find recurring issues or to validate that updates to the profiles and algorithms are working as intended. In short, the logging and compliance module 930 provides transparency and accountability for the cobot’s operation, ensuring that every action is traceable and that the system can be continually improved.

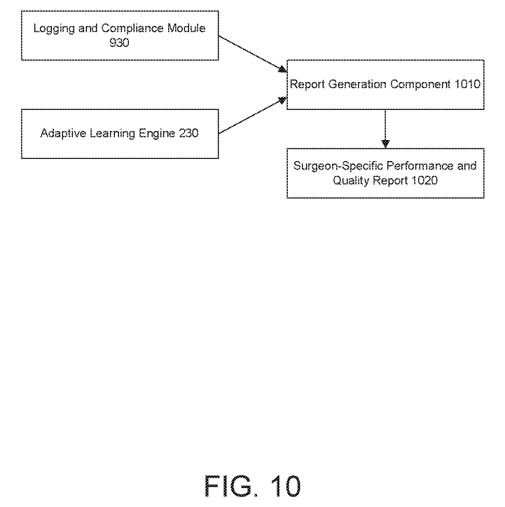

FIG. 10 illustrates how the system aggregates data into useful reports following surgical procedures. A report generation component 1010 compiles the logged data and analytical results related to the surgeon’s workflow, producing a surgeon-specific performance and quality report 1020. Such a report can include:

Workflow Efficiency Metrics: For example, the total duration of the procedure and each of its phases, the time intervals between instrument requests and deliveries, and any idle times or delays encountered. By comparing these metrics to the expected timeline from the surgeon’s profile, the report can highlight where the workflow was optimal and where improvements might be made.

Instrument Utilization Statistics: The report enumerates the instruments used during the procedure, how many times each instrument was handed off, and any instruments that were prepared but ultimately not used. These statistics help in fine-tuning instrument tray preparation, identifying tools that might be unnecessary for certain procedures or ensuring frequently used tools are given priority placement.

Adaptive Adjustments and Overrides: The report notes instances of dynamic adjustments, such as any on-the-fly changes the cobot made via context-awareness (for example, automatically bringing an extra instrument when a deviation was detected) or any manual overrides by the staff. Documenting these events provides insight into how well the system’s automated workflow aligned with actual practice and where the surgeon or team had to step in.

Safety and Compliance Record: The report includes a summary of safety-related events, confirming whether the surgery proceeded without any safety interventions (or detailing any that occurred). It might list if any proximity warnings were triggered or if an emergency stop was used, along with verification that sterile field integrity was maintained throughout. This serves as a quality assurance log for each case.

All these metrics and observations are presented in an organized format (e.g., as a digital dashboard or a printed report) that allows the surgeon and OR management to review the cobot’s performance in the context of that surgery. The reports not only provide feedback to the individual surgeon, helping them understand, for instance, if their cases tend to have more instrument wait time than average, but also inform the ongoing optimization of the system. Over time, aggregated reports across many operations can be analyzed to improve both the surgeon-specific profiles and the overall system configuration, thereby contributing to evidence-driven enhancements in surgical workflows and patient care.

What is claimed is:

1. An integrated perioperative cobotic system for surgeon-specific workflow management, the system comprising:
a configurable cobotic platform comprising a base, at least one articulated robotic arm with a tool handling interface, and one or more sensors configured for interaction with a surgical environment;
a surgeon-specific workflow engine configured to access a stored workflow profile for an identified surgeon and to direct the cobotic platform according to surgeon-specific preferences;
an instrument handling and pre-positioning module configured to manage surgical instruments in accordance with the identified surgeon’s workflow profile;
an adaptive learning system configured to refine the workflow profile based on data collected from multiple surgical procedures performed by the surgeon;
an electronic medical record and operating room scheduling integration interface configured to retrieve surgeon preference data for a scheduled surgical procedure and adjust the workflow profile based on the retrieved data;
a context-aware task execution system configured to respond to surgeon inputs and real-time surgical conditions to perform automated tasks according to the workflow profile;
a user interface configured to allow the surgeon to review and customize the workflow profile;
one or more safety mechanisms including an emergency override control to allow manual intervention and controls to ensure compliance with sterile-field protocols; and
a data analytics and reporting module configured to collect usage data and generate performance reports.

2. The system of claim 1, wherein the configurable cobotic platform is mobile or semi-fixed and includes the articulated robotic arm and sensors arranged to dynamically interact with the surgical environment, the cobotic platform being configured to autonomously navigate within an operating room while avoiding sterile field zones and adapting to real-time procedural changes.

3. The system of claim 1, wherein the surgeon-specific workflow engine stores a personalized workflow profile for each surgeon, including the surgeon’s instrument preferences, procedural sequencing, instrument handoff timing, and ergonomic positioning preferences, and wherein the workflow engine is configured to automatically retrieve and apply the workflow profile linked to a surgeon’s identifier at the start of a surgical procedure.

4. The system of claim 1, wherein the instrument handling and pre-positioning module is configured to prepare and arrange surgical instruments prior to and during surgery based on surgeon-specific usage patterns and anticipated procedural phases, including adjusting an instrument tray layout, positioning the cobotic platform or robotic arm, and orienting instrument handoffs according to the surgeon’s dominant hand and preferred reach zone.

5. The system of claim 1, wherein the adaptive learning and workflow refinement system is configured to learn from each surgical procedure performed by the surgeon and continuously refine the associated workflow profile’s timing of actions, instrument sequence accuracy, and predictive readiness, the refinement being based on both automated observations of the surgeon’s intraoperative behavior and explicit feedback provided by the surgeon.

6. The system of claim 1, wherein the electronic medical record and operating room scheduling integration interface is configured to interface with electronic medical records and operating room scheduling systems to pre-load surgeon-specific preferences for the scheduled procedure, including preferences tailored to the procedure type, patient context, and expected case duration, and to dynamically update the workflow profile in response to surgical plan changes, intraoperative deviations, or patient-specific considerations.

7. The system of claim 1, wherein the context-aware task execution system is configured to detect surgeon inputs comprising voice commands, hand gestures, or footswitch signals and, in response, trigger corresponding cobotic actions including adjusting a camera view, swapping a surgical instrument, or providing retraction assistance, and wherein the task execution system modifies the timing and sequencing of tasks based on real-time detection of the surgical phase and environmental cues in the operating room.

8. The system of claim 1, wherein the user interface for profile customization is configured to present the surgeon’s workflow preferences and allow the surgeon to view and adjust the preferences prior to the surgical procedure, the user interface supporting global default settings as well as procedure-specific customizations and requiring surgeon approval of any changes to the workflow profile.

9. The system of claim 1, wherein the safety, override, and compliance features comprise an emergency override control to immediately halt or modify cobotic actions, role-based access controls restricting user permissions to edit surgeon workflow profiles, and automated enforcement of sterile-protocol compliance during all cobotic operations, and wherein the system is further configured to track and log all workflow actions and interventions for quality improvement, credentialing, and postoperative analysis.

10. The system of claim 1, wherein the data analytics and reporting module is configured to capture surgeon-specific efficiency metrics including surgical instrument utilization rates and procedural timing data, and to generate analytical reports based on the captured metrics for review by hospital administration and surgical quality improvement teams.

11. A method of facilitating a surgeon-specific surgical workflow using a perioperative cobotic system, the method comprising:
identifying a surgeon associated with a scheduled surgical procedure via a surgeon-specific identifier;
retrieving a personalized workflow profile corresponding to the identified surgeon from a data store;
configuring a cobotic platform based on the retrieved workflow profile by pre-positioning surgical instruments and adjusting at least one robotic arm according to the surgeon’s preferences before a start of the surgical procedure;
monitoring, during the surgical procedure, inputs from the surgeon and a progression of the procedure using sensors of the cobotic system;
executing context-aware cobotic actions in response to the surgeon’s inputs and the surgical progression in accordance with the workflow profile;
dynamically adjusting timing or sequencing of the cobotic actions based on real-time changes or deviations in the surgical procedure; and
updating the surgeon’s workflow profile after the procedure based on data collected during the procedure.

12. The method of claim 11, wherein retrieving the personalized workflow profile comprises accessing surgeon preference information from at least one of an electronic medical record system or an operating room scheduling system corresponding to the scheduled procedure, and using the accessed information to select or configure the workflow profile for the surgeon.

13. The method of claim 11, wherein monitoring inputs from the surgeon includes recognizing voice commands, hand gestures, or footswitch signals, and wherein executing the context-aware cobotic actions includes performing corresponding tasks including adjusting a camera angle, swapping a surgical instrument, or providing retraction assistance during the procedure.

14. The method of claim 11, further comprising logging operational metrics of the surgical procedure, including surgeon-specific instrument utilization frequencies and durations of procedural steps, and generating a report based on the logged metrics for review by hospital administration or a surgical quality improvement team.

15. The method of claim 11, further comprising receiving post-procedure feedback from the surgeon and incorporating the feedback into the updating of the surgeon’s workflow profile to refine future surgical workflow preferences for the surgeon.

16. The method of claim 11, wherein configuring the cobotic platform based on the workflow profile includes ensuring the cobotic platform remains outside sterile-field boundaries during instrument positioning, and wherein executing the context-aware cobotic actions is automatically halted or overridden upon receiving an emergency stop command from operating room personnel.

17. A surgeon-specific workflow engine for a perioperative cobotic system, the workflow engine comprising:
a memory storing a plurality of surgeon-specific workflow profiles, each profile including instrument preferences, procedural sequencing, and ergonomic settings for a particular surgeon;
an identification interface configured to receive surgeon identification data and automatically select the corresponding workflow profile from the memory;
one or more processors communicatively coupled to the memory and the identification interface, the one or more processors being configured to execute the selected workflow profile by generating control signals for the perioperative cobotic platform to carry out tasks according to the surgeon’s specified preferences.

18. The surgeon-specific workflow engine of claim 17, wherein the identification interface comprises at least one of a radio-frequency identification reader or a biometric scanner that, upon detecting the surgeon’s identity, triggers automatic loading of the surgeon’s workflow profile.

19. The surgeon-specific workflow engine of claim 17, wherein the one or more processors are further configured to update and refine the stored workflow profiles over time by incorporating data from completed surgical procedures and explicit surgeon feedback, thereby learning and adjusting to the surgeon’s evolving preferences.

20. The surgeon-specific workflow engine of claim 17, wherein the one or more processors are configured to communicate with hospital information systems including electronic medical records and operating room scheduling systems to adjust or select the workflow profile based on patient-specific or case-specific information.
